(19) United States
(12) Patent Application Publication
(10) Pub. No.: US 2026/0015633 A1
(43) Pub. Date: Jan. 15, 2026

(54) PROCESS FOR PRODUCTION OF ALCOHOLS FROM CELL LYSATE

(71) Applicant: Gevo, Inc., Englewood, CO (US)

(72) Inventors: Kent Evans, Englewood, CO (US); Paul Bloom, Englewood, CO (US); Jenifer J. Meinholz, Parker, CO (US); Madison Perry, Denver, CO (US); Nicholas Tumminaro, Denver, CO (US); Mattias Greer, Louisville, CO (US); Matthew Marzo, Denver, CO (US)

(21) Appl. No.: 19/265,763

(22) Filed: Jul. 10, 2025

Related U.S. Application Data
(60) Provisional application No. 63/669,613, filed on Jul. 10, 2024.

(51) Int. Cl. C12P 7/16; C12N 9/88; C12N 15/81

(52) U.S. Cl. CPC C12P 7/16; C12N 9/88; C12N 15/81; C12Y 401/01001

(57) ABSTRACT

Disclosed are systems and methods for producing renewable alcohols by fermentative processes using cell lysate or a combination of cell lysate and whole cells, as well as the cell lysate itself. The disclosure also provides processes and systems for converting renewable alcohols to transportation fuels, such as, gasoline, jet fuel, and diesel fuels.

### CROSS-REFERENCE TO RELATED APPLICATIONS

This application claims the benefit of U.S. Provisional Patent Application No. 63/669,613, filed Jul. 10, 2024, the disclosure of which is incorporated herein by reference in its entirety and to which priority is claimed.

### FIELD

This disclosure relates to systems and methods for producing renewable alcohols by fermentative processes using cell lysate or a combination of cell lysate and whole cells.

### BACKGROUND

Alcohols are important industrial chemicals, useful as reagents, solvents, fuel additives, feedstock chemicals in the plastics industry, and chemical intermediates. Renewable alcohols may be used in the production of transportation fuels, such as, gasoline, jet fuel, and diesel fuels. Accordingly, there is a high demand for alcohols, for example, butanol, as well as for efficient and environmentally friendly methods of producing the alcohols.

Production of alcohol, such as ethanol and butanol, using fermentation by microorganisms is one such environmentally friendly production method. In order to develop an economically competitive fermentation process, a number of factors such as, the development of a microorganism (“biocatalyst”) that can produce the alcohol, carbon sources capable of being metabolized by the microorganism, recovery of the alcohol from a fermentation broth, co-product formation, and the potential for contamination may all be considered in the development of this process. In particular, the production of alcohol using fermentation by a microorganism may be limited by the toxicity of the alcohol to the microorganism.

The accumulation of the alcohol during fermentation may be toxic to the microorganism and potentially impact the performance of the microorganism. That is, the end product (e.g., alcohol) of the fermentation may inhibit the growth rate and production rate of the microorganism and may result in lower cell densities. Thus, alcohol production may be limited in whole cell-based fermentation, due to the need to maintain cell viability. One option to address toxicity is the dilution of the fermentation broth, for example, by the addition of water. However, this additional water load in the fermentation process may reduce capital productivity and may require further handling and processing including the need for additional equipment such as fermenters, pumps, mixing tanks, storage tanks, heat exchangers, distillation columns, and the like as well as additional costs. Another option to address toxicity is the removal of the alcohol from the fermentation vessel as it is being produced. For example, in situ product removal (ISPR) (also referred to as extractive fermentation) can be used to remove alcohol from the fermentation vessel as it is produced, thereby allowing the microorganism to produce alcohol at higher yields. However, extractive fermentation may increase the capital costs, operating costs, and overall energy usage of the alcohol production process. Another option to address toxicity is using cell components involved in the fermentation pathway, rather than whole cells, to eliminate constraints related to maintaining cell viability.

Isobutanol production using a synthetic biochemistry pathway for the conversion of glucose to isobutanol is known. This synthetic approach involves the expression and purification of fourteen enzymes that participate in the conversion of glucose to isobutanol, as well as the combination of these sixteen enzymes with appropriate cofactors at appropriate levels. The complexity and cost associated with this synthetic approach may be a barrier to commercialization. A method for producing bioethanol using a mixture of yeast cell lysate and saccharification enzyme to simultaneously saccharify starch to produce glucose and ferment the glucose is also known. The yeast cells used in this method as well as the conditions of the method are selected and optimized for simultaneous saccharification and fermentation. For example, the simultaneous saccharification and fermentation are performed at an increased temperature to accommodate saccharification enzymes. The yeast cells and conditions of this method may thus be contraindicated for fermenting alcohol from other carbon sources or for fermenting alcohols other than ethanol.

Thus, there is a continuing need to address the challenge of alcohol toxicity to microorganisms used for microbial fermentation and to develop more efficient methods and systems for producing alcohols, such as butanol.

### SUMMARY

This disclosure describes, among other things, methods of producing renewable alcohol and compositions for producing renewable alcohol, particularly isobutanol, as well as methods for converting the renewable alcohol to fuel. The disclosure also describes recombinant yeast cell lysates for producing isobutanol and methods for producing the recombinant yeast cell lysates.

In some embodiments, a method for producing renewable alcohol includes: a) providing a recombinant yeast cell comprising an alcohol producing metabolic pathway; b) mixing the recombinant yeast cell with a source of glucose to produce a first reaction mixture and fermenting the first reaction mixture to produce alcohol; c) recovering the alcohol; d) harvesting the recombinant yeast cell; e) disrupting the harvested recombinant yeast cell to obtain a substantially cell-free cell lysate; (f) mixing the substantially cell-free cell lysate with a source of glucose to produce a second reaction mixture and fermenting the second reaction mixture to produce alcohol; and (g) recovering the alcohol.

In some embodiments, a method for producing renewable alcohol includes: a) providing a recombinant yeast cell comprising an alcohol producing metabolic pathway; b) disrupting the recombinant yeast cell to obtain a substantially cell-free cell lysate; (c) mixing the substantially cell-free cell lysate with a source of glucose to produce a reaction mixture and fermenting the reaction mixture to produce alcohol; and (d) recovering the alcohol.

In some embodiments, a composition for producing isobutanol includes: a) a recombinant yeast cell lysate and, b) optionally, a buffer, where the recombinant yeast cell lysate comprises: i) an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof; and ii) a fragment of recombinant yeast cell wall, a fragment of recombinant yeast cell membrane, or combinations thereof. The recombinant yeast cell lysate may also comprise various endogenously produced substances, such as endogenously expressed proteins, including cytosolic proteins. A multiphase fermentation composition including a) an aqueous phase comprising the composition and a source of glucose; and b) an organic phase comprising isobutanol and an organic solvent may be formed. The organic solvent may comprise oleyl alcohol and/or other biocompatible organic solvents. The composition may be used in a method for producing isobutanol by mixing the composition with a source of glucose to produce a reaction mixture; b) fermenting the reaction mixture to produce isobutanol; and c) recovering the isobutanol. The recombinant yeast cell lysate may be selected from the group consisting of recombinant Saccharomyces cell lysate, recombinant Kluyveromyces cell lysate, recombinant Candida cell lysate, recombinant Pichia cell lysate, recombinant Issatchenkia cell lysate, recombinant Debaryomyces cell lysate, recombinant Hansenula cell lysate, recombinant Yarrowia cell lysate, recombinant Schizosaccharomyces cell lysate, and combinations thereof.

In some embodiments, a recombinant yeast cell lysate for producing isobutanol is provided, the recombinant yeast cell lysate made by a method including: (a) providing a recombinant yeast cell comprising an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof; b) mixing the recombinant yeast cell with a source of glucose to produce a first reaction mixture and fermenting the first reaction mixture to produce isobutanol; c) optionally, recovering the isobutanol; d) harvesting the recombinant yeast cell; and e) disrupting the harvested recombinant yeast cell to obtain the recombinant yeast cell lysate. The recombinant yeast cell may be mechanically disrupted in step e) via homogenization, cavitation, via one or more cycles of freeze-thawing, or a combination thereof.

In some embodiments, a method for producing a recombinant yeast cell lysate includes: (a) providing a recombinant yeast cell comprising an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof; b) mixing the recombinant yeast cell with a source of glucose to produce a first reaction mixture and fermenting the first reaction mixture to produce isobutanol; c) optionally, recovering the isobutanol; d) harvesting the recombinant yeast cell; and e) disrupting the harvested recombinant yeast cell to obtain the recombinant yeast cell lysate. The recombinant yeast cell may be mechanically disrupted in step e) via homogenization, cavitation, via one or more cycles of freeze-thawing, or a combination thereof.

In some embodiments, one or more of the following features may be included in any feasible combination in any of the described methods or compositions. For example, the step of recovering the alcohol may include (i) combining the reaction mixture (for example, the first reaction mixture or the second reaction mixture) and the alcohol with an organic solvent, wherein the alcohol partitions into an organic phase and the reaction mixture (for example, the first reaction mixture or the second reaction mixture) partitions into an aqueous phase; and (ii) distilling the organic phase to separate the alcohol. The alcohol may be recovered before the concentration of the alcohol reaches a level toxic to the recombinant yeast cell. The organic solvent may comprise oleyl alcohol, fatty acids, fatty acid methyl esters, or a combination thereof.

In some embodiments, the alcohol producing metabolic pathway of step (a) may comprise an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

In some embodiments, the recombinant yeast cell may be mechanically disrupted in step (b) via homogenization, cavitation, or a combination thereof.

In some embodiments, the recombinant yeast cell may be mechanically disrupted in step (b) via one or more cycles of freeze-thawing.

In some embodiments, the recombinant yeast cell may be selected from the group consisting of Saccharomyces, Kluyveromyces, Candida, Pichia, Issatchenkia, Debaryomyces, Hansenula, Yarrowia, Schizosaccharomyces, and combinations thereof.

In some embodiments, the renewable alcohol may be selected from the group consisting of ethanol, 1-butanol, 2-butanol, isobutanol, tert-butanol, and combinations thereof.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the embodiments disclosed herein. It should also be appreciated that terminology explicitly employed herein that also may appear in any disclosure incorporated by reference should be accorded with a meaning most consistent with the particular concepts disclosed herein.

### BRIEF DESCRIPTION OF THE DRAWINGS

FIG. 1 is a graph depicting the isobutanol yield of whole cell fermentation, cell lysate fermentation, and cell lysate fermentation with added organic solvent.

FIG. 2 is a graph depicting the effect of different organic solvents on the isobutanol yield of cell lysate fermentation.

### DETAILED DESCRIPTION

Unless defined otherwise, all technical and scientific terms used herein have the same meaning as commonly understood by one of ordinary skill in the art to which this invention belongs. In case of conflict, the present application, including the definitions, will control. Also, unless otherwise required by context, singular terms shall include pluralities, and plural terms shall include the singular. All publications, patents and other references mentioned herein are incorporated by reference in their entireties for all purposes.

In order to further define this invention, the following terms and definitions are herein provided.

Reference throughout this specification to “some embodiments”, “one embodiment” or “an embodiment” means a particular feature, structure or characteristic described in connection with the embodiment is included in at least one embodiment. Thus, the appearances of the phrases “in some embodiments”, “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

As used herein, “recombinant microorganism” refers to a microorganism, such as bacterium or yeast, that is modified by use of recombinant DNA techniques, for example, by engineering a host cell to comprise a biosynthetic pathway, such as a biosynthetic pathway to produce an alcohol (e.g., butanol).

As used herein, “fermentable carbon source” refers to a carbon source capable of being metabolized by the microorganisms disclosed herein for the production of alcohol. Suitable fermentable carbon sources include, but are not limited to, monosaccharides such as glucose or fructose; disaccharides such as lactose or sucrose; oligosaccharides; polysaccharides such as starch or cellulose; C₅ sugars such as xylose and arabinose; carbon substrates such as methane; and mixtures thereof. The carbon source may be derived from biomass. The terms “fermentable carbon source”, “carbon substrate”, and “fermentable carbon substrate” are used interchangeably.

As used herein, the term “feedstock” refers to a feed in a fermentation process, the feed containing a fermentable carbon source with or without undissolved solids, and where applicable, the feed containing the fermentable carbon source before or after the fermentable carbon source has been liberated from starch or obtained from the breakdown of complex sugars by further processing, such as by liquefaction, saccharification, or other processes. Feedstock includes or may be derived from biomass. Suitable feedstocks include, but are not limited to, rye, wheat, barley, corn, corn mash, cane, cane mash, cellulosic material, lignocellulosic material, and mixtures thereof.

The terms “fermentation broth”, “fermentation liquid”, “fermentation medium”, and “fermented mixture” are used interchangeably and refer to a mixture containing, among other constituents, fermentative microorganisms, fermentable carbon sources (e.g., glucose), and alcohol.

Disclosed herein is a process for producing renewable alcohol using microbial fermentation. Renewable alcohols include, but are not limited to, C₁ to C₈ alkyl alcohols, isomers of C₁ to C₈ alkyl alcohols, or mixtures thereof. In some embodiments, the alcohols are C₂ to C₈ alkyl alcohols. In some embodiments, the alcohols are C₂ to C₈ alkyl alcohols or C₃ to C₆ alkyl alcohols. It will be appreciated that C₁ to C₈ alkyl alcohols include, but are not limited to, methanol, ethanol, propanol, butanol, pentanol, and hexanol. C₂ to C₈ alkyl alcohols include, but are not limited to, ethanol, propanol, butanol, and pentanol.

In order to develop an economically viable microbial fermentation process, a number of factors, such as, the development or identification of a microorganism (“biocatalyst”) that can produce the alcohol, the identification of carbon sources capable of being metabolized by the microorganism, the efficient recovery of the alcohol from a fermentation broth, co-product formation, and the potential for contamination, may be considered.

### Fermentative Recombinant Microorganism

The process for producing renewable alcohol described herein may use any alcohol-producing microorganism, including recombinant alcohol-producing microorganisms. Though many native or naturally occurring alcohol-producing microorganisms (e.g., bacteria, yeast) exist, such native microorganisms may not produce alcohol at a high enough yield for commercialization. For example, Clostridium acetobutylicum and other Clostridia are known to produce butanol via fermentation. In fact, acetone-butanol-ethanol (ABE) fermentation by Clostridium acetobutylicum is one of the oldest known industrial fermentations (as are also the pathways and genes responsible for the production of the acetone, butanol, and ethanol). Production of butanol by the ABE process, however, is limited by the toxic effect of the butanol on Clostridium acetobutylicum and by the generation of co-products, such as acetone and ethanol. Genetic modification using known molecular biological techniques can be used to develop recombinant microorganisms that fermentatively produce alcohol, including butanol, at higher yields. Suitable microorganisms that may be genetically modified include bacteria, cyanobacteria, filamentous fungi, or yeast.

Recombinant microorganisms may be engineered to express a selected metabolic pathway and/or to produce a desired metabolite, such as alcohol, to reduce or eliminate the production of undesired co-products, and/or to otherwise increase the yield of the desired metabolite, such as alcohol (e.g., butanol). Metabolites include starting materials (e.g., glucose or pyruvate), intermediates (e.g., 2-ketoisovalerate, acetaldehyde), and end products (e.g., ethanol, butanol, other alcohols) of a selected metabolic pathway. Metabolites can be used to construct more complex molecules or broken down into simpler molecules. Intermediate metabolites may be synthesized from other metabolites, used to make more complex molecules, or broken down into simpler molecules, often with the release of chemical energy. Exemplary metabolites include glucose, pyruvate, and alcohol, such as butanol.

Recombinant microorganisms that produce alcohol are known in the art (e.g., Ohta et al., Appl. Environ. Microbiol. 57:893-900 (1991); Underwood et al., Appl. Environ. Microbiol. 68:1071-81 (2002); Shen and Liao, Metab. Eng. 10:312-20 (2008); Hahnai et al., Appl. Environ. Microbiol. 73:7814-8 (2007); U.S. Pat. Nos. 5,514,583; 5,712,133; International Publication No. WO 1995/028476; Feldmann et al., Appl. Microbiol. Biotechnol. 38:354-61 (1992); Zhang et al., Science 267:240-3 (1995); U.S. Patent Publication No. 2007/0031918A1; U.S. Pat. Nos. 7,223,575; 7,741,119; U.S. Patent Publication No. 2009/0203099A1; U.S. Patent Publication No. 2009/0246846A1; and International Publication No. WO 2010/075241, which are herein incorporated by reference.

For example, the metabolic pathways of microorganisms may be genetically modified to produce butanol. The metabolite butanol may be produced by a recombinant microorganism metabolically engineered to express or over-express a metabolic pathway that converts pyruvate to butanol. The metabolic pathway may also be modified to reduce or eliminate undesired metabolites or co-products, thereby improving the yield of alcohol. The production of butanol by a recombinant microorganism is disclosed in, for example, U.S. Pat. Nos. 7,851,188; 7,993,889; 8,178,328; 8,206,970; and 9,790,521; and U.S. Patent Application Publication Nos. 2007/0292927; 2008/0182308; 2008/0274525; 2009/0305363; 2009/0305370; 2011/0250610; 2011/0313206; 2011/0111472; and 2012/0258873, which are herein incorporated by reference. Recombinant microorganisms that produce butanol at higher yields are disclosed in U.S. Pat. No. 8,455,239 and International Publication No. WO 2010/05125, which are herein incorporated by reference.

In some embodiments, the recombinant microorganism comprises a butanol biosynthetic pathway or a biosynthetic pathway for butanol isomers, such as 1-butanol, 2-butanol, or isobutanol. In some embodiments, the biosynthetic pathway converts pyruvate to a fermentative product. In some embodiments, the biosynthetic pathway converts pyruvate as well as amino acids to a fermentative product. In some embodiments, at least one, at least two, at least three, or at least four polypeptides catalyzing substrate to product conversions of a pathway are encoded by heterologous polynucleotides in the microorganism. In some embodiments, all polypeptides catalyzing substrate to product conversions of a pathway are encoded by heterologous polynucleotides in the microorganism.

Suitable microorganisms capable of producing alcohol (e.g., butanol) via a biosynthetic pathway include members of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Paenibacillus, Arthrobacter, Corynebacterium, Brevibacterium, Schizosaccharomyces, Kluveromyces, Yarrowia, Pichia, Zygosaccharomyces, Debaryomyces, Candida, Brettanomyces, Pachysolen, Hansenula, Issatchenkia, Trichosporon, Yamadazyma, or Saccharomyces. In some embodiments, the recombinant microorganisms may be selected from the group consisting of Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, Candida sonorensis, Candida methanosorbosa, Kluyveromyces lactis, Kluyveromyces marxianus, Kluveromyces thermotolerans, Issatchenkia orientalis, Debaryomyces hansenii, and Saccharomyces cerevisiae.

In some embodiments, the genetically modified microorganism is a recombinant yeast. In some embodiments, the genetically modified microorganism is a crabtree-positive recombinant yeast. Suitable yeast include Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus, Saccharomyces mikitae, Saccharomyces paradoxus, Saccharomyces uvarum, Saccharomyces castelli, Zygosaccharomyces rouxii, Zygosaccharomyces bailli, and Candida glabrata.

In some embodiments, the host cell is Saccharomyces cerevisiae. Saccharomyces cerevisiae is known in the art and available from a variety of sources including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961. A recombinant host cell comprising an “engineered alcohol production pathway” (such as an engineered butanol or isobutanol production pathway) refers to a host cell containing a modified pathway that produces alcohol in a manner different than that normally present in the host cell. Such differences include production of alcohol not typically produced by the host cell or increased or more efficient production.

Biosynthetic pathways for the production of isobutanol that may be used include those described in U.S. Pat. Nos. 7,851,188; 7,993,388; and International Publication No. WO 2007/050671, which are incorporated herein by reference. In some embodiments, the isobutanol biosynthetic pathway may comprise the following substrate to product conversions:
a) pyruvate to acetolactate, which may be catalyzed by, for example, acetolactate synthase;
b) the acetolactate from step a) to 2,3-dihydroxyisovalerate, which may be catalyzed by, for example, acetohydroxy acid reductoisomerase;
c) the 2,3-dihydroxyisovalerate from step b) to α-ketoisovalerate, which may be catalyzed by, for example, acetohydroxy acid dehydratase;
d) the α-ketoisovalerate from step c) to isobutyraldehyde, which may be catalyzed by, for example, a branched-chain α-keto acid decarboxylase; and,
e) the isobutyraldehyde from step d) to isobutanol, which may be catalyzed by, for example, a branched-chain alcohol dehydrogenase.

In some embodiments, the isobutanol biosynthetic pathway may comprise the following substrate to product conversions:
a) pyruvate to acetolactate, which may be catalyzed by, for example, acetolactate synthase;
b) the acetolactate from step a) to 2,3-dihydroxyisovalerate, which may be catalyzed by, for example, ketol-acid reductoisomerase;
c) the 2,3-dihydroxyisovalerate from step b) to α-ketoisovalerate, which may be catalyzed by, for example, dihydroxyacid dehydratase;
d) the α-ketoisovalerate from step c) to valine, which may be catalyzed by, for example, transaminase or valine dehydrogenase;
e) the valine from step d) to isobutylamine, which may be catalyzed by, for example, valine decarboxylase;
f) the isobutylamine from step e) to isobutyraldehyde, which may be catalyzed by, for example, omega transaminase; and,
g) the isobutyraldehyde from step f) to isobutanol, which may be catalyzed by, for example, a branched-chain alcohol dehydrogenase.

In some embodiments, the isobutanol biosynthetic pathway may comprise the following substrate to product conversions:
a) pyruvate to acetolactate, which may be catalyzed by, for example, acetolactate synthase;
b) the acetolactate from step a) to 2,3-dihydroxyisovalerate, which may be catalyzed by, for example, acetohydroxy acid reductoisomerase;
c) the 2,3-dihydroxyisovalerate from step b) to α-ketoisovalerate, which may be catalyzed by, for example, acetohydroxy acid dehydratase;
d) the α-ketoisovalerate from step c) to isobutyryl-CoA, which may be catalyzed by, for example, branched-chain keto acid dehydrogenase;
e) the isobutyryl-CoA from step d) to isobutyraldehyde, which may be catalyzed by, for example, acylating aldehyde dehydrogenase; and,
f) the isobutyraldehyde from step e) to isobutanol, which may be catalyzed by, for example, a branched-chain alcohol dehydrogenase.

Biosynthetic pathways for the production of 1-butanol that may be used include those described in U.S. Patent Application Publication No. 2008/0182308 and International Publication No. WO 2007/041269, which are incorporated herein by reference. In some embodiments, the 1-butanol biosynthetic pathway may comprise the following substrate to product conversions:
a) acetyl-CoA to acetoacetyl-CoA, which may be catalyzed by, for example, acetyl-CoA acetyltransferase;
b) the acetoacetyl-CoA from step a) to 3-hydroxybutyryl-CoA, which may be catalyzed by, for example, 3-hydroxybutyryl-CoA dehydrogenase;
c) the 3-hydroxybutyryl-CoA from step b) to crotonyl-CoA, which may be catalyzed by, for example, crotonase;
d) the crotonyl-CoA from step c) to butyryl-CoA, which may be catalyzed by, for example, butyryl-CoA dehydrogenase;
e) the butyryl-CoA from step d) to butyraldehyde, which may be catalyzed by, for example, butyraldehyde dehydrogenase; and,
f) the butyraldehyde from step e) to 1-butanol, which may be catalyzed by, for example, butanol dehydrogenase.

Biosynthetic pathways for the production of 2-butanol that may be used include those described in U.S. Pat. No. 8,206,970; U.S. Patent Application Publication Nos. 2007/0292927 and 2009/0155870; International Publication Nos. WO 2007/130518 and WO 2007/130521, which are incorporated herein by reference. In some embodiments, the 2-butanol biosynthetic pathway may comprise the following substrate to product conversions:
a) pyruvate to alpha-acetolactate, which may be catalyzed by, for example, acetolactate synthase;
b) the alpha-acetolactate from step a) to acetoin, which may be catalyzed by, for example, acetolactate decarboxylase;
c) the acetoin from step b) to 3-amino-2-butanol, which may be catalyzed by, for example, acetonin aminase;
d) the 3-amino-2-butanol from step c) to 3-amino-2-butanol phosphate, which may be catalyzed by, for example, aminobutanol kinase;
e) the 3-amino-2-butanol phosphate from step d) to 2-butanone, which may be catalyzed by, for example, aminobutanol phosphate phosphorylase; and,
f) the 2-butanone from step e) to 2-butanol, which may be catalyzed by, for example, butanol dehydrogenase.

In some embodiments, the 2-butanol biosynthetic pathway may comprise the following substrate to product conversions:
a) pyruvate to alpha-acetolactate, which may be catalyzed by, for example, acetolactate synthase;
b) the alpha-acetolactate from step a) to acetoin, which may be catalyzed by, for example, acetolactate decarboxylase;
c) the acetoin to 2,3-butanediol from step b), which may be catalyzed by, for example, butanediol dehydrogenase;
d) the 2,3-butanediol from step c) to 2-butanone, which may be catalyzed by, for example, dial dehydratase; and,
e) the 2-butanone from step d) to 2-butanol, which may be catalyzed by, for example, butanol dehydrogenase.

Biosynthetic pathways for the production of 2-butanone that may be used include those described in U.S. Pat. No. 8,206,970 and U.S. Patent Application Publication Nos. 2007/0292927 and 2009/0155870, which are incorporated herein by reference. In some embodiment, the 2-butanone biosynthetic pathway may comprise the following substrate to product conversions:
a) pyruvate to alpha-acetolactate, which may be catalyzed by, for example, acetolactate synthase;
b) the alpha-acetolactate from step a) to acetoin, which may be catalyzed by, for example, acetolactate decarboxylase;
c) the acetoin from step b) to 3-amino-2-butanol, which may be catalyzed by, for example, acetonin aminase;
d) the 3-amino-2-butanol from step c) to 3-amino-2-butanol phosphate, which may be catalyzed by, for example, aminobutanol kinase; and,
e) the 3-amino-2-butanol phosphate from step d) to 2-butanone, which may be catalyzed by, for example, aminobutanol phosphate phosphorylase.

In some embodiments, the 2-butanone biosynthetic pathway may comprise the following substrate to product conversions:
a) pyruvate to alpha-acetolactate, which may be catalyzed by, for example, acetolactate synthase;
b) the alpha-acetolactate from step a) to acetoin which may be catalyzed by, for example, acetolactate decarboxylase;
c) the acetoin from step b) to 2,3-butanediol, which may be catalyzed by, for example, butanediol dehydrogenase;
d) the 2,3-butanediol from step c) to 2-butanone, which may be catalyzed by, for example, diol dehydratase.

The terms “acetohydroxyacid synthase,” “acetolactate synthase,” and “acetolactate synthetase” (abbreviated “ALS”) are used interchangeably herein to refer to an enzyme that catalyzes the conversion of pyruvate to acetolactate and CO₂. Example acetolactate synthases are known by the EC number 2.2.1.6 (Enzyme Nomenclature 1992, Academic Press, San Diego). These enzymes are available from a number of sources, including, but not limited to, Bacillus subtilis (GenBank Nos: CAB07802.1, Z99122, NCBI (National Center for Biotechnology Information) amino acid sequence, NCBI nucleotide sequence, respectively), CAB15618, Klebsiella pneumoniae (GenBank Nos: AAA25079, M73842), and Lactococcus lactis (GenBank Nos: AAA25161, L16975)

The term “ketol-acid reductoisomerase” (“KARI”), “acetohydroxy acid isomeroreductase,” and “acetohydroxy acid reductoisomerase” will be used interchangeably and refer to enzymes capable of catalyzing the reaction of (S)-acetolactate to 2,3-dihydroxyisovalerate. Example KARI enzymes may be classified as EC number EC 1.1.1.86 (Enzyme Nomenclature 1992, Academic Press, San Diego) and are available from a vast array of microorganisms, including, but not limited to, Escherichia coli (GenBank Nos: NP_418222, NC_000913), Saccharomyces cerevisiae (GenBank Nos: NP_013459, NC_001144), Methanococcus maripaludis (GenBank Nos: CAF30210, BX957220), Bacillus subtilis (GenBank Nos: CAB14789, Z99118), and Anaerostipes caccae. Ketol-acid reductoisomerase (KARI) enzymes are described in U.S. Pat. Nos. 7,910,342; 8,129,162; and 9,512,408; U.S. Patent Application Publication Nos. 2008/0261230; 2009/0163376; 2010/0197519; and International Publication Nos. WO 2011/041415 and WO 2012/129555, which are incorporated herein by reference. Examples of suitable KARIs include those from Lactococcus lactis, Vibrio cholera, Pseudomonas aeruginosa PAO1, and Pseudomonas fluorescens PF5 mutants. In some embodiments, the KARI uses NADH or NADPH.

The term “acetohydroxy acid dehydratase” and “dihydroxyacid dehydratase” (“DHAD”) refers to an enzyme that catalyzes the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, E. coli (GenBank Nos: YP_026248, NC000913), Saccharomyces cerevisiae (GenBank Nos: NP_012550, NC_001142), M. maripaludis (GenBank Nos: CAF29874, BX957219), B. subtilis (GenBank Nos: CAB14105, Z99115), L. lactis, and N. crassa. U.S. Patent Application Publication No. 2010/0081154; U.S. Pat. Nos. 7,851,188; and 8,241,878, which are incorporated herein by reference in their entireties, describe dihydroxyacid dehydratases (DHADs), including a DHAD from Streptococcus mutans and variants thereof.

The term “branched-chain α-keto acid decarboxylase,” “α-ketoacid decarboxylase,” “α-ketoisovalerate decarboxylase,” or “2-ketoisovalerate decarboxylase” (“KIVD”) refers to an enzyme that catalyzes the conversion of α-ketoisovalerate to isobutyraldehyde and CO₂. Example branched-chain α-keto acid decarboxylases are known by the EC number 4.1.1.72 and are available from a number of sources, including, but not limited to, Lactococcus lactis (GenBank Nos: AAS49166, AY548760; CAG34226, AJ746364), Salmonella typhimurium (GenBank Nos: NP_461346, NC_003197), Clostridium acetobutylicum (GenBank Nos: NP_149189, NC_001988), M. caseolyticus, and L. grayi.

The term “branched-chain alcohol dehydrogenase” (“ADH”) refers to an enzyme that catalyzes the conversion of isobutyraldehyde to isobutanol. Example branched-chain alcohol dehydrogenases are known by the EC number 1.1.1.265 but may also be classified under other alcohol dehydrogenases (specifically, EC 1.1.1.1 or 1.1.1.2). Alcohol dehydrogenases may be NADPH-dependent or NADH-dependent. Such enzymes are available from a number of sources, including, but not limited to, S. cerevisiae (GenBank Nos: NP_010656, NC_001136, NP_014051, NC_001145), E. coli (GenBank Nos: NP_417484, NC_000913), and C. acetobutylicum (GenBank Nos: NP_349892, NC_003030; NP_349891, NC_003030). U.S. Patent Application Publication No. 2009/0269823, which is incorporated herein by reference in its entirety, describes SadB, an alcohol dehydrogenase (ADH) from Achromobacter xylosoxidans. Alcohol dehydrogenases can also include horse liver ADH and Beijerinkia indica ADH, as described by U.S. Patent Application Publication No. 2011/0269199, which is incorporated herein by reference in its entirety.

The term “butanol dehydrogenase” refers to a polypeptide (or polypeptides) having enzyme activity that catalyzes the conversion of isobutyraldehyde to isobutanol or the conversion of 2-butanone and 2-butanol. Butanol dehydrogenases are a subset of a broad family of alcohol dehydrogenases. Butanol dehydrogenase may be NAD-dependent or NADP-dependent. The NAD-dependent enzymes are known as EC 1.1.1.1 and are available from, for example, Rhodococcus ruber (GenBank Nos: CAD36475, AJ491307). The NADP-dependent enzymes are known as EC 1.1.1.2 and are available from, for example, Pyrococcus furiosus (GenBank Nos: AAC25556, AF013169). Additionally, a butanol dehydrogenase is available from E. coli (GenBank Nos: NP_417484, NC_000913) and a cyclohexanol dehydrogenase is available from Acinetobacter species (GenBank Nos: AAG10026, AF282240). The term “butanol dehydrogenase” also refers to an enzyme that catalyzes the conversion of butyraldehyde to 1-butanol, using either NADH or NADPH as a cofactor. Butanol dehydrogenases are available from, for example, C. acetobutylicum (GenBank NOs: NP_149325, NC_001988; NP_349891, NC_003030, NP_349892, and NC_003030) and E. coli (GenBank NOs: NP_417-484 and NC_000913).

The term “branched-chain keto acid dehydrogenase” refers to an enzyme that catalyzes the conversion of α-ketoisovalerate to isobutyryl-CoA (isobutyryl-coenzyme A), typically using NAD+ as an electron acceptor. Example branched-chain keto acid dehydrogenases are known by the EC number 1.2.4.4. Such branched-chain keto acid dehydrogenases are comprised of four subunits and are available from a vast array of microorganisms, including, but not limited to, B. subtilis (GenBank Nos: CAB14336, Z99116; CAB14335, Z99116; CAB14334, Z99116; and CAB14337, Z99116) and Pseudomonas putida (GenBank Nos: AAA65614, M57613; AAA65615, M57613; AAA65617, M57613; and AAA65618, M57613).

The term “pyruvate decarboxylase” refers to an enzyme that catalyzes the decarboxylation of pyruvic acid to acetaldehyde and CO₂. Pyruvate dehydrogenases are known by the EC number 4.1.1.1. These enzymes are found in a number of yeast, including Saccharomyces cerevisiae (GenBank Nos: CAA97575, CAA97705, CAA97091).

Host cells comprising an isobutanol biosynthetic pathway as provided herein may further comprise one or more additional modifications. U.S. Patent Application Publication No. 2009/0305363, which is incorporated herein by reference in its entirety, discloses increased conversion of pyruvate to acetolactate by engineering yeast for expression of a cytosol-localized acetolactate synthase and substantial elimination of pyruvate decarboxylase activity. In some embodiments, the host cells comprise modifications to reduce glycerol-3-phosphate dehydrogenase activity, to disrupt at least one gene encoding a polypeptide having pyruvate decarboxylase activity, and/or to disrupt at least one gene encoding a regulatory element controlling pyruvate decarboxylase gene expression, as described in U.S. Patent Application Publication No. 2009/0305363, which is incorporated herein by reference in its entirety. Modifications to a host cell that provide for increased carbon flux through an Entner-Doudoroff Pathway or reducing equivalents balance are described in U.S. Patent Application Publication No. 2010/0120105, which is incorporated herein by reference in its entirety. Other modifications include integration of at least one polynucleotide encoding a polypeptide that catalyzes a step in a pyruvate-using biosynthetic pathway.

Other modifications include at least one deletion, mutation, and/or substitution in an endogenous polynucleotide encoding a polypeptide having acetolactate reductase activity. As used herein, “acetolactate reductase activity” refers to the activity of any polypeptide having the ability to catalyze the conversion of acetolactate to 2,3-dihydroxy-2-methyl butyrate (DHMB). DHMB includes “fast DHMB,” which has the 2S, 3S configuration, and “slow DHMB,” which has the 2S, 3R configuration (See Kaneko et al., Phytochemistry 39:115-120 (1995), which refers to “fast DHMB” as anglyceric acid and “slow DHMB” as tiglyceric acid). In some embodiments, the polypeptide having acetolactate reductase activity is YMR226C of Saccharomyces cerevisiae or a homolog thereof.

Additional modifications include a deletion, mutation, and/or substitution in an endogenous polynucleotide encoding a polypeptide having aldehyde dehydrogenase and/or aldehyde oxidase activity, including a polypeptide that catalyzes the oxidation (dehydrogenation) of aldehydes, a polypeptide that catalyzes the conversion of isobutyraldehyde to isobutyric acid, and a polypeptide that corresponds to Enzyme Commission Numbers EC 1.2.1.3, EC 1.2.1.4 or EC 1.2.1.5. Aldehyde oxidases include a polypeptide that catalyzes production of carboxylic acids from aldehydes. Such polypeptides include a polypeptide that catalyzes the conversion of isobutyraldehyde to isobutyric acid and a polypeptide that corresponds to Enzyme Commission Number EC 1.2.3.1. In some embodiments, the polypeptide having aldehyde dehydrogenase activity is ALD6 from Saccharomyces cerevisiae or a homolog thereof.

A genetic modification which has the effect of reducing glucose repression in a PDC-yeast host cell is disclosed in U.S. Patent Application Publication No. 2011/0124060, incorporated herein by reference. The term “PDC-” refers to a cell that has a genetic modification to inactivate or reduce expression of at least one gene encoding pyruvate decarboxylase (PDC), such that the cell substantially or completely lacks pyruvate decarboxylase enzyme activity. If the yeast cell has more than one expressed (active) PDC gene, then each of the active PDC genes may be inactivated or have minimal expression, thereby producing a PDC-cell. In some embodiments, the pyruvate decarboxylase that is deleted or downregulated is selected from the group consisting of: PDC1, PDC5, PDC6, and combinations thereof. In some embodiments, the pyruvate decarboxylase is selected from PDC1 pyruvate decarboxylase from Saccharomyces cerevisiae, PDC5 pyruvate decarboxylase from Saccharomyces cerevisiae, PDC6 pyruvate decarboxylase from Saccharomyces cerevisiae, pyruvate decarboxylase from Candida glabrata, PDC1 pyruvate decarboxylase from Pichia stipites, PDC2 pyruvate decarboxylase from Pichia stipites, pyruvate decarboxylase from Kluveromyces lactis, pyruvate decarboxylase from Yarrowia lipolytica, pyruvate decarboxylase from Schizosaccharomyces pombe, and pyruvate decarboxylase from Zygosaccharomyces rouxii. In some embodiments, host cells contain a deletion or down-regulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

In an isobutanologen (PDC−) strain, PDC is deleted, the pyruvate dehydrogenase complex (PDH) pathway remains intact, and isobutanol production pathway enzymes are introduced. Often, the first enzyme to act in the isobutanol production pathway is acetolactate synthase (ALS). In isobutanologens, the carbon flux distribution for biomass growth and for the isobutanol pathway under aerobic conditions depends on the relative activity of ALS instead of the PDH enzyme. The physiological behavior of a recombinant isobutanologen is different from an unmodified S. cerevisiae due to the effect of the deletion of PDC genes and introduction of heterologous isobutanol pathway enzymes. To maximize biomass production in a recombinant isobutanologen in aerobic growth phase, the carbon flux has to channel through the PDH pathway efficiently to improve biomass yield and minimize carbon flux to isobutanol pathway leakages. Pathway leakage products can include isobutanol and isobutyric acid, which can adversely affect biomass growth rate and the final biomass achieved. In the production phase, the isobutanol yield and productivity can be adversely affected by accumulation of pathway intermediates (e.g., glycerol and isobutyric acid). Thus, the optimal operating regime (growth and production) for an ethanologen may not be the optimal operating regime for an isobutanologen.

WIPO publication number WO 2011/103300 discloses recombinant host cells comprising (a) at least one heterologous polynucleotide encoding a polypeptide having dihydroxy-acid dehydratase activity; and (b) (i) at least one deletion, mutation, and/or substitution in an endogenous gene encoding a polypeptide affecting Fe—S cluster biosynthesis; and/or (b) (ii) at least one heterologous polynucleotide encoding a polypeptide affecting Fe—S cluster biosynthesis. In some embodiments, the polypeptide affecting Fe—S cluster biosynthesis is encoded by AFT1, AFT2, FRA2, GRX3, or CCC1.

Additionally, host cells may comprise heterologous polynucleotides encoding a polypeptide with phosphoketolase activity and/or a heterologous polynucleotide encoding a polypeptide with phosphotransacetylase activity.

Also, given that accumulation of a alcohol during fermentation may be toxic to some microorganisms, as discussed above, recombinant microorganisms may be engineered to have a greater tolerance for alcohol. Examples of genetic modifications that may improve tolerance to alcohols include, but are not limited to, expression and/or modifications of relA, spoT, and dksA genes (described in U.S. Patent Application Publication No. 2009/0203139, incorporated herein by reference), elongase genes (Yazawa, et al., Appl. Microbiol. Biotechnol. 91:1593-1600, 2011), heat shock proteins (HSPs), as well as genes associated with lipid and fatty acid metabolism and cell membrane composition (see, e.g. Ma, et al., Appl. Microbiol. Biotechnol. 87:829-845, 2010). In addition, recombinant microorganisms may be engineered to have a certain level of thermotolerance, for example, by genetically modifying the microorganism to express stress-related genes, such as the genes encoding proteins involved in the ubiquitination process.

In some embodiments, any particular nucleic acid molecule or polypeptide may be at least 80%, 85%, 90%, 95%, 96%, 97%, 98%, or 99% identical to a nucleotide sequence or polypeptide sequence described herein. The term “percent identity”, as known in the art, is a relationship between two or more polypeptide sequences or two or more polynucleotide sequences, as determined by comparing the sequences. “Identity” may also refer to the degree of sequence relatedness between polypeptide or polynucleotide sequences, as determined by the match between strings of such sequences. “Identity” and “similarity” can be readily calculated by known methods, including but not limited to those disclosed in Computational Molecular Biology (Lesk, A. M., Ed.) Oxford University: NY (1988); Biocomputing: Informatics and Genome Projects (Smith, D. W., Ed.) Academic: NY (1993); Computer Analysis of Sequence Data, Part I (Griffin, A. M., and Griffin, H. G., Eds.) Humania: NJ (1994); Sequence Analysis in Molecular Biology (von Heinje, G., Ed.) Academic (1987); and Sequence Analysis Primer (Gribskov, M. and Devereux, J., Eds.) Stockton: NY (1991).

Standard recombinant DNA and molecular cloning techniques are well known in the art and are described by Sambrook, et al. (Sambrook, J., Fritsch, E. F. and Maniatis, T. (Molecular Cloning: A Laboratory Manual; Cold Spring Harbor Laboratory Press, Cold Spring Harbor, 1989, here in referred to as Maniatis) and by Ausubel, et al. (Ausubel, et al., Current Protocols in Molecular Biology, published by Greene Publishing Assoc. and Wiley-Interscience, 1987). Examples of methods to construct microorganisms that comprise a butanol biosynthetic pathway are disclosed, for example, in U.S. Pat. No. 7,851,188 and U.S. Patent Application Publication Nos. 2007/0092957; 2007/0259410; 2007/0292927; 2008/0182308; 2008/0274525; 2009/0155870; 2009/0305363; and 2009/0305370, each of which is herein incorporated by reference.

Growth and/or Fermentation Media and Carbon Sources

Recombinant microorganism whole cells and cell lysate (e.g., whole yeast cells and yeast cell lysate) disclosed herein are contacted with suitable carbon sources, typically in fermentation media. Suitable carbon sources may include monosaccharides, such as fructose or glucose, disaccharides, oligosaccharides, such as lactose, maltose, galactose, or sucrose, polysaccharides, such as starch or cellulose, ethanol, lactate, succinate, glycerol, carbon dioxide, methanol, glucose, dextrose, fructose, lactose, sucrose, xylose, arabinose, dextrose, cellulose, methane, amino acids, or mixtures thereof. Suitable carbon sources may also include unpurified mixtures from renewable feedstocks, such as cheese whey permeate, cornsteep liquor, sugar beet molasses, and barley malt. The source of carbon used in the present disclosure may encompass a wide variety of carbon containing substrates and may be selected based on a number of factors, such as the type of microorganism used or whether whole cells or cell lysate is used. The source of carbon may affect the activity of fermentation enzymes and may be selected to enhance the activity of fermentation enzymes. Without being bound by theory, it is believed that fermentation enzymes may become active or more active, when cells are grown in the presence of dextrose and/or when cell lysate derived from such cells undergoes fermentation in the presence of dextrose.

In some embodiments, the carbon source is selected from glucose, fructose, sucrose, and mixtures thereof, including mixtures thereof with C5 sugars, such as xylose and/or arabinose, for yeasts cells modified to use C5 sugars. Sucrose may be derived from renewable sugar sources, such as sugar cane, sugar beets, cassava, sweet sorghum, or mixtures thereof. Glucose and dextrose may be derived from renewable grain sources via saccharification of starch-based feedstocks, including grains, such as corn, wheat, rye, barley, oats, or mixtures thereof. In addition, fermentable sugars may be derived from renewable cellulosic or lignocellulosic biomass via processes of pretreatment and saccharification, as described, for example, in U.S. Patent Application Publication No. 2007/0031918, which is herein incorporated by reference.

Biomass, when used in reference to carbon source, refers to any cellulosic or lignocellulosic material and includes materials comprising cellulose and, optionally, further comprising hemicellulose, lignin, starch, oligosaccharides, and/or monosaccharides. Biomass can also comprise additional components, such as protein and/or lipid. Biomass may be derived from a single source, or biomass may comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, or mixtures thereof.

In addition to an appropriate carbon source, the growth and/or fermentation media may contain additional components, such as suitable minerals, salts, cofactors, buffers and other components, known to those skilled in the art, suitable for the growth of the cultures and promotion of a selected enzymatic pathway. Like the carbon source, these additional components may affect fermentation and alcohol production and may be selected to increase alcohol production. For example, the growth and/or fermentation media may include a select buffer(s) at a selected concentration(s) to maximize alcohol production. Without being bound by theory, it is believed that the alcohol production of the cell lysate may be increased by using a buffer including sodium phosphate, MgSO₄, one or more protease inhibitors, dithiothreitol, and glucose in the cell lysate fermentation medium.

Optionally, the fermentation media may contain ethanol. In some embodiments, when a recombinant microorganism having a butanol biosynthetic pathway is used as microorganism for butanol production, supplementation of the fermentation medium with a 2-carbon substrate (e.g., ethanol) may facilitate the survival and growth of the recombinant microorganism. Thus, in some embodiments, ethanol may be supplied to the fermentation medium.

Suitable growth and/or fermentation media, for whole cells and cell lysate (e.g., whole yeast cells and yeast cell lysate), include common commercially prepared media such as Luria Bertani (LB) broth, Sabouraud Dextrose (SD) broth, or Yeast Medium (YM) broth or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth and/or fermentation media can also be used, such as YPEG medium, a blend of peptone, yeast extract, ethanol, and glycerol, or DM1U medium, a blend of mineral, salts, and dextrose in optimal proportions, can also be used. Media deriving from agricultural sources such as corn thin stillage and corn mash may also be used in growth and/or fermentation. The use of agents known to modulate catabolite repression directly or indirectly, e.g., cyclic adenosine 2′,3′-monophosphate (cAMP), may also be incorporated into the growth and/or fermentation medium.

Growth and/or Fermentation Conditions

Typically, growth and/or fermentation of cells (e.g., whole yeast cells) occurs at a temperature in the range of about 20° C. to about 40° C., or about 25° C. to about 35° C., in an appropriate medium. In some embodiments, growth and/or fermentation of cells occurs at a temperature of about 20° C., about 22° C., about 25° C., about 28° C., about 30° C., about 32° C., about 35° C., about 37° C., or about 40° C. In some aspects, growth and/or fermentation of cells occurs at a temperature of about 20° C. to about 35° C., or about 20° C. to about 30° C., or about 25° C. to about 30° C. Certain cells are more thermo-tolerant and, in such cells, growth and/or fermentation may occur at higher temperatures, such as about 42° C., 45° C., 47° C., and even above 50° C. (e.g., for short periods of time).

Cell lysate (e.g., yeast cell lysate) fermentation may occur at a temperature in the range of about 20° C. to about 45° C., or about 28° C. to about 42° C., in an appropriate medium. In some embodiments, cell lysate fermentation occurs at a temperature of about 20° C., about 22° C., about 25° C., about 27° C., about 30° C., about 32° C., about 35° C., about 37° C., or about 42° C. In some aspects, cell lysate fermentation occurs at a temperature of about 30° C. to about 45° C., or about 30° C. to about 42° C., or about 32° C. to about 42° C. Certain cells are more thermo-tolerant and, in cell lysates derived from such cells, fermentation may occur at higher temperatures, such as about 42° C., 45° C., 47° C., and even above 50° C. (e.g., for short periods of time).

Suitable pH for the growth and/or fermentation of cells (e.g., whole yeast cells) may range from about pH 3.0 to about pH 9.0, or about pH 3.0 to about pH 7.5. In some embodiments, the pH for the growth and/or fermentation of cells ranges from about pH 4.0 to about pH 9.0, or about 4.0 to about 8.0. In some aspects, a pH range of about pH 4.0 to about pH 7.0, or about pH 4.0 to about pH 6.0, or about pH 4.5 to about pH 5.5, is used for the fermentation of whole cells. Suitable pH for cell lysate fermentation (e.g., yeast cell lysate) may range from about pH 3.0 to about pH 9.0, or about pH 4.0 to about pH 9.0, or about 4.0 to about 8.0. In some examples, a pH range of about pH 5.0 to about pH 8.0, or about pH 6.0 to about pH 8.0, or about pH 6.5 to about pH 7.5 is used for the fermentation of cell lysate.

Fermentations may be performed under aerobic or anaerobic conditions. In some embodiments, anaerobic or microaerobic conditions are used for fermentation. Cell lysate fermentation may be performed under anaerobic conditions.

### Microorganism Cell Disruption

Cell disruption or lysis and methods of disrupting or lysing cells are known. Various cell lysis methods have been developed and used at both laboratory scale and industrial scale. Specialized lysis equipment, including sonicators and homogenizers, and chemicals for lysing cells, including reagents, enzymes, and surfactants, are commercially available. There are numerous methods for performing cell lysis. These methods may generally include mechanical methods, such as homogenization, mortar and pestle manual grinding, and bead beating, and non-mechanical methods, such as physical, chemical, and biological methods. Physical methods include thermal lysis (e.g., freeze/thaw cycling), osmotic shock, and cavitation. Chemical methods include alkaline lysis and detergent lysis, and biological methods include enzymatic lysis.

Homogenization, which may employ a high-pressure homogenizer (HPH), is commonly used for large scale microbial cell disruption. In homogenization, cells may be forced through an orifice valve using high pressure and lysis may occur due to high shear force at the orifice, as a cell is subjected to compression in the orifice and expands once discharged from the orifice. Sonication, which may employ a sonicator, involves exposing cells to high frequency sound waves that disrupt and lyse the cells. Sonication is generally used for cell volumes of less than 100 mL. Cavitation involves the formation and subsequent rupture of cavities or bubbles, which may be formed by reducing local pressure. Ultrasonic or hydrodynamic methods may be used to generate cavitation by ultrasonic vibration or by increasing velocity, respectively. In ultrasonic cavitation, an ultrasonic vibration generates a sound wave that creates pressure changes, while hydrodynamic cavitation is produced by pumping the cell suspension through a constricted channel, resulting in an increase in velocity. Thermal lysis may be performed by repeated freezing and thawing cycles, which results in the formation of ice on the cell membrane and the consequent breakdown of the cell membrane.

The cell lysis method may be selected based on many factors, including the type of microorganism, the desired end product of the lysis, the scale (commercial-scale versus lab- or bench-scale), and lysis efficiency. For example, E. coli cells comprise a cytoplasmic membrane, a cell wall, and an outer membrane, while yeast cells include cell walls. Some microorganism cells may thus be easier to disrupt than others. In some examples, enzymatic lysis may be employed to remove cell walls and the enzyme may be selected based on the properties of the cell wall. The desired end product of the lysis is also an important consideration, and, in some cases, the lysis method may be selected to minimize damage to delicate end products. For example, chemical lysis methods may adversely affect the end product.

In some embodiments, the microorganism cells disclosed herein (e.g., recombinant yeast cell) may be is mechanically disrupted via homogenization, bead beating, or a combination thereof, preferably homogenization.

In some embodiments, the microorganism cells disclosed herein (e.g., recombinant yeast cell) may be is physically disrupted via heating (e.g., freeze/thaw cycling), osmotic shock, cavitation, or a combination thereof, preferably cavitation.

In some embodiments, the microorganism cells disclosed herein (e.g., recombinant yeast cell) may be is chemically disrupted via alkaline lysis and/or detergent lysis. In some embodiments, the microorganism cells disclosed herein (e.g., recombinant yeast cell) may be biologically disrupted via enzymatic lysis.

In some embodiments, the microorganism cells disclosed herein (e.g., recombinant yeast cell) may be disrupted via a lysis method selected from the group consisting of homogenization, bead beating, heating (e.g., freeze/thaw cycling), osmotic shock, cavitation, alkaline lysis, detergent lysis, enzymatic lysis, and combinations thereof.

Regardless of the cell disruption or lysis method used, fermentation enzymes in the cell lysate may optionally be isolated or separated from cell debris after cell lysis. Preferably, fermentation enzymes in the cell lysate are not isolated or separated from cell debris after cell lysis. Without being bound by theory, it is believed that isolation or separation of fermentation enzymes from cell debris after cell lysis may result in the loss of fermentation enzymes. If the separation of fermentation enzymes from cell debris after cell lysis can be avoided, the complexity and cost of the entire process may also be reduced.

### Industrial Batch and Continuous Fermentations

Butanol and other alcohols may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable and comprise a typical batch system with the exception that the carbon source or carbon substrate is added in increments as the fermentation progresses. Fed-batch systems are useful when catabolite repression is likely to inhibit the metabolism of the cells and/or when it is desirable to have limited amounts of the carbon source or carbon substrate in the media. Batch and fed-batch fermentations are common and well known in the art and examples can be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Butanol or other alcohol products may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density, where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology.

It is contemplated that the production of butanol or other alcohol products can be practiced using batch, fed-batch, and/or continuous processes and that any known mode of fermentation may be suitable. Additionally, it is contemplated that cells can be immobilized on a substrate as whole cell catalysts and subjected to fermentation conditions for butanol production.

Methods for Recovering Alcohol from the Fermentation Medium

The bioproduced and/or renewable alcohol, e.g., butanol, may be isolated from the fermentation medium using methods known in the art. For example, solids may be removed from the fermentation medium by centrifugation, filtration, decantation, or the like. Isobutanol may be isolated from the fermentation medium, which may optionally be pre-treated to remove solids as described above, using methods such as distillation (e.g., vacuum distillation), azeotropic distillation, liquid-liquid extraction, adsorption, gas stripping, membrane evaporation or other membrane-based separations, or pervaporation.

Gas stripping may be done by passing a gas such as air, nitrogen, or carbon dioxide through the fermentation medium, thereby forming an butanol-containing gas phase. The butanol may be recovered from the butanol-containing gas phase using methods known in the art, such as using a chilled water trap to condense the alcohol or scrubbing the gas phase with a solvent.

Butanol, e.g., isobutanol, may form a low boiling point, azeotropic mixture with water, and distillation may be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer.

The butanol-water mixture may form a heterogeneous azeotrope so that distillation may be used in combination with decantation to isolate and purify the butanol (e.g., isobutanol). In this method, the butanol-containing fermentation broth is distilled to near the azeotropic composition. Then, the azeotropic mixture is condensed, and the butanol is separated from the fermentation medium by decantation. The decanted aqueous phase may be returned to the first distillation column as reflux. The butanol-rich decanted organic phase may be further purified by distillation in a second distillation column.

The butanol (e.g., isobutanol) may also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption may also be used to isolate butanol (e.g., isobutanol) from the fermentation medium. In this method, the fermentation broth containing the butanol is distilled to near the azeotropic composition and then the remaining water is removed by use of an adsorbent, such as a molecular sieve.

Additionally, distillation in combination with pervaporation may be used to isolate and purify the butanol (e.g., isobutanol) from the fermentation medium. In this method, the fermentation broth containing the butanol is distilled to near the azeotropic composition, and then the remaining water is removed by pervaporation through a hydrophilic membrane.

In situ product removal (ISPR) (also referred to as extractive fermentation) can be used to remove butanol (or other fermentative alcohol) from the fermentation vessel as it is produced, thereby allowing the microorganism to produce butanol at high yields. ISPR can be carried out in a batch mode, where a volume of organic extractant is added to the fermentation vessel and the extractant is not removed during the process, or a continuous mode, where product is continually removed from the reactor. In some instances, the ISPR utilizes liquid-liquid extraction. For example, the fermentation medium, which includes the microorganism, may be contacted with an organic extractant (typically before the butanol concentration reaches a toxic level), and the organic extractant and the fermentation medium may form a biphasic mixture of an aqueous phase comprising the fermentation medium and a non-aqueous organic phase comprising the butanol. The butanol may partition into the organic phase, decreasing the concentration in the aqueous phase containing the microorganism and thereby limiting the exposure of the microorganism to the inhibitory butanol.

The butanol-containing organic phase may be separated from the aqueous phase using methods known in the art, including but not limited to, siphoning, decantation, centrifugation, using a gravity settler, membrane-assisted phase splitting, and the like. Recovery of the butanol from the butanol-containing organic phase can be done using methods known in the art, including but not limited to, distillation, adsorption by resins, separation by molecular sieves, pervaporation, and the like. Specifically, distillation may be used to recover the butanol from the butanol-containing organic phase. The extractant may be recycled to the butanol production and/or recovery process.

Extractive fermentation may be carried out in a continuous mode in a stirred tank fermenter. In this mode, the biphasic mixture is removed from the fermenter and the two phases are separated by means known in the art as described above. After separation, the fermentation medium may be recycled to the fermenter or may be replaced with fresh medium. Then, the extractant may be treated to recover the alcohol product, e.g., butanol, as described above. The extractant may then be recycled back into the fermenter for further extraction of the product. Alternatively, fresh extractant may be continuously added to the fermenter to replace the removed extractant. This continuous mode of operation offers several advantages. Because the product is continually removed from the reactor, a smaller volume of organic extractant composition is required, which enables a larger volume of the fermentation medium to be used, resulting in higher production yields.

A batch fermentation mode may also be used. Batch fermentation, which is well known in the art, is a closed system in which the composition of the fermentation medium is set at the beginning of the fermentation and is not subjected to artificial alterations during the process. In this mode, a volume of organic extractant composition is added to the fermenter and the extractant is not removed during the process. The organic extractant composition may comprise more than one solvent and may be formed in the fermenter by separate addition of a first solvent and a second solvent. Alternatively, the organic extractant composition may comprise more than one solvent and the first and second solvents may be combined to form the extractant composition prior to the addition of the extractant composition to the fermenter. Although this mode is simpler than the continuous or the entirely continuous modes described above, it requires a larger volume of organic extractant composition to minimize the concentration of the inhibitory alcohol product, e.g., butanol, in the fermentation medium. Consequently, the volume of the fermentation medium is less, and the amount of product produced is less than that obtained using the continuous mode.

Fed-batch fermentation mode may also be used. Fed-batch fermentation is a variation of the standard batch system, in which the nutrients, for example glucose, are added in increments during the fermentation. The amount and the rate of addition of the nutrient may be determined by routine experimentation. For example, the concentration of critical nutrients in the fermentation medium may be monitored during the fermentation. Alternatively, more easily measured factors such as pH, dissolved oxygen, and the partial pressure of waste gases, such as carbon dioxide, may be monitored. From these measured parameters, the rate of nutrient addition may be determined. The amount of organic extractant composition used and its methods of addition in this mode may be the same as in the batchwise mode, described above.

Extraction of the alcohol product may occur downstream of the fermenter, such that the extraction of the alcohol product, e.g., butanol, via contact with the organic extractant composition is carried out on the fermentation medium removed from the fermenter, or in situ. The fermentation medium may be removed from the fermenter continuously or periodically and the extraction of the alcohol product, e.g., butanol, by the organic extractant composition may be done with or without the removal of the recombinant microorganisms from the fermentation medium. The recombinant microorganisms may be removed from the fermentation medium by means known in the art including, but not limited to, filtration or centrifugation. After separation of the fermentation medium from the extractant, as described above, the fermentation medium may be recycled into the fermenter, discarded, or treated for the removal of any remaining alcohol product, e.g., butanol. Similarly, the isolated recombinant microorganisms may also be recycled into the fermenter. After treatment to recover the alcohol product, e.g., butanol, the organic extractant composition may be recycled for use in the extraction process. Alternatively, fresh organic extractant composition may be used, whereby toxicity of the extractant composition to the microorganisms may present less of a problem. If the microorganisms are separated from the fermentation medium before contacting with the extractant composition, the problem of extractant toxicity may be further reduced. Furthermore, by extracting the product in this way, there is less chance of forming an emulsion and evaporation of the extractant is minimized, alleviating potential environmental concerns.

The volume of the organic extractant to be used depends on a number of factors, including the volume of the fermentation medium, the size of the fermenter, the partition coefficient of the extractant for the alcohol, e.g., butanol, and the fermentation mode chosen, as described below. The volume of the organic extractant composition may be about 3% to about 60% of the fermenter working volume. In some instances, the volume of the organic extractant composition may be about 3% to about 50% of the fermenter working volume, or about 3% to about 20% of the fermenter working volume; or about 3% to about 10% of the fermenter working volume. In some instances, in a batchwise mode, the volume of the organic extractant composition may be 20% to about 60% of the fermenter working volume or 30% to about 60% of the fermenter working volume.

It may be beneficial to use the smallest concentration of extractant in the fermenter as possible to maximize the volume of the aqueous phase, and therefore, the amount of fermentative microorganisms in the fermenter. The process may be operated in an entirely continuous mode, in which the extractant is continuously recycled between the fermenter and a separation apparatus and the fermentation medium is continuously removed from the fermenter and replenished with fresh medium. In this entirely continuous mode, the alcohol product, e.g., butanol, is not allowed to reach the critical toxic concentration and fresh nutrients are continuously provided so that the fermentation may be carried out for long periods of time. The apparatuses that may be used to carry out these modes of two-phase extractive fermentations are well known in the art.

The ratio of the extractant to the fermentation medium may be from about 1:20 to about 20:1 on a volume:volume basis, or from about 1:15 to about 15:1, or from about 1:12 to about 12:1, or from about 1:10 to about 10:1, or from about 1:9 to about 9:1, or from about 1:8 to about 8:1 on a volume:volume basis.

In extractive fermentation, the organic extractant may be water immiscible. The organic extractant or organic extractant composition may comprise a solvent (e.g., organic solvent). Suitable solvents include C4 to C22 fatty alcohols, C4 to C28 fatty acids, esters of C4 to C28 fatty acids, C4 to C22 fatty aldehydes, C7 to C22 ethers, amides, phosphate esters, ureas, phenols (phenolics), phosphinates, carbamates, phosphoramide, or mixtures thereof. The solvent may be selected from the group consisting of oleyl alcohol, phenyl alcohol, cetyl alcohol, lauryl alcohol, myristyl alcohol, stearyl alcohol, oleic acid, lauric acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, isododecanol, lauric aldehyde, 2-methylundecanal, oleamide, linoleamide, palmitamide, stearylamide, 2-ethyl-1-hexanol, 2-hexyl-1-decanol, 2-octyl-1-dodecanol, octanol (e.g., 1-octanol), heptanol, phenetole, and mixtures thereof. The solvent may be polar and/or may include one or more of a phosphorous atom, a nitrogen atom, a sulfur atom, or an oxygen atom. In some embodiments, the organic extractant composition or the organic phase of the biphasic mixture may contain ethanol. The solvent may exhibit hydrogen bonding.

In certain embodiments, the organic extractant composition may comprise a first solvent and a second solvent. In some embodiments, where more than one solvent is used for the extraction, the solvents may first be combined together before contacting the fermentation medium. For example, a first solvent and a second solvent may be combined in a vessel, such as a mixing tank, to form the extractant composition, which may then be added to a vessel containing the fermentation medium (e.g., fermentation vessel). Alternatively, the first and second solvents need not be combined before contacting the fermentation medium and may instead be combined at the same time as contacting the fermentation medium. For example, the first and second solvents may be separately added, simultaneously or at different times, to a vessel that contains the fermentation medium. In some embodiments, contacting the fermentation medium with the organic extractant composition comprises contacting the fermentation medium with the first solvent and then contacting the fermentation medium and the first solvent with the second solvent. In some embodiments, the contacting with the second solvent occurs in the same vessel as the contacting with the first solvent. In some embodiments, the contacting with the second solvent occurs in a different vessel than the contacting with the first solvent. For example, the first solvent may be contacted with the fermentation medium in a first vessel and the contents of the first vessel may then be transferred to second vessel, in which contact with the second solvent occurs.

The organic extractant composition may contact the fermentation medium at the start of the fermentation. Alternatively, the organic extractant composition may contact the fermentation medium after the microorganism has achieved a desired level of growth, which can be determined by measuring the optical density of the culture. Further, the organic extractant composition may contact the fermentation medium at a time at which the alcohol, e.g., butanol, level in the fermentation medium reaches a preselected level, for example, before the alcohol, e.g., butanol, concentration reaches a level that is toxic to the fermentative microorganism. The alcohol, e.g., butanol, concentration may be monitored during the fermentation using methods known in the art, such as by gas chromatography or high performance liquid chromatography.

Fermentations may be performed under aerobic or anaerobic conditions. In some embodiments, anaerobic or microaerobic conditions are used for fermentation. Fermentation may be run under aerobic conditions for a time sufficient for the culture to achieve a preselected level of growth. An inducer may optionally be added to induce the expression of the alcohol, e.g., butanol, biosynthetic pathway in the recombinant or modified microorganism, and fermentation conditions may be switched to microaerobic or anaerobic conditions to stimulate butanol production.

### Further Processing of Renewable Alcohol

Renewable alcohols, such as ethanol and isobutanol, may be sold as commodity chemicals directly. Alternatively, renewable alcohols may be further processed, for example, dehydrated to their respective olefins (e.g. ethylene and isobutene and one or more renewable linear butenes-typically a mixture of isobutene, 1-butene and cis/trans-2-butene). The renewable ethylene and renewable butenes can then also either be sold directly or still further processed (e.g., separated or reacted) in a variety of different ways to produce a wide variety of renewable hydrocarbon product streams. In some embodiments, further processing may comprise mixing the renewable ethylene and/or butene with ethylene and/or butylene produced by conventional methods (e.g., petroleum cracking) to produce an array of hydrocarbon compounds comprising renewable carbon. Accordingly, such compounds, while not composed solely of renewable carbon, still comprise at least some renewable carbon, with concomitant environmental advantages.

In some embodiments, renewable butene may be produced via the dehydration of renewable isobutanol. The renewable butene formed thereby is typically a tunable mixture of butene isomers, which is easily separated from the isobutanol feed to the dehydration reaction, and can be sold directly as a mixture, reacted as a mixture to form other hydrocarbons (e.g., polybutenes), or the mixture of renewable butene isomers can be separated (e.g., by distillation, by selective conversion, etc.) into individual butene isomers, which can then either be sold individually as feedstocks, polymerized (e.g. to renewable polyisobutylene or butene copolymers), oligomerized (e.g., dimerized, trimerized, etc.) to form higher molecular weight olefins (e.g. isooctene or pentamethylheptenes), isomerized (e.g. isobutene isomerized to linear butenes, 1-butene isomerized to 2-butene, or 2-butene isomerized to 1-butene, etc.), dehydrogenated (e.g. to butadiene), as well as combinations of such processes. In particular, isobutene dimers and trimers can be hydrogenated to provide, e.g., renewable isooctane, which may be useful as a renewable transportation fuel or a renewable additive for transportation fuels.

In some embodiments, renewable ethylene may be produced via the dehydration of renewable ethanol. The renewable ethylene produced thereby is generally of very high purity and is easily separated from the unreacted feedstock of the dehydration reaction. The renewable ethylene can then be either sold directly as a feedstock, or subsequently converted to higher value renewable hydrocarbons, such as higher molecular weight olefins produced by oligomerization reactions (e.g. dimers, trimers, etc.), polymerized to form renewable polyethylene, oxidized form renewable ethylene oxide (which can be subsequently be polymerized to form renewable polyethylene oxide, or converted to other renewable polyethylene oxide derivatives), converted to dichloroethane (for subsequent conversion to vinyl chloride and polymerization thereof), or used as a renewable feedstock for alkylating other olefins or aromatics (e.g., alkylation of benzene to produce ethylbenzene).

### EXAMPLES

The following non-limiting examples will further illustrate the systems, methods, and compositions disclosed herein. It should be understood that, while the following examples involve glucose as the carbon source, other carbon sources, feedstocks, or biomass sources, such as corn, may be used for feedstock without departing from the present invention. Moreover, while the following examples involve butanol, other alcohols may be produced without departing from the present invention. It should also be understood that the experimental test methods described below, including the concentrations specified in the experimental test methods, are adapted for bench- or lab-scale production. The experimental test methods described below may be scaled up for commercial/industrial and/or pilot production.

The volume of seed fermenter culture and medium may be increased for a pilot-scale or an industrial-scale fermenter. Also, alternative methods of cell preparation may be used for increased quantities of cell mass, for example, using mechanical cell disruption equipment rather than manual grinding, to produce greater volumes of cell lysate. Whole cells need not be frozen prior to disruption using mechanical cell disruption equipment and other large scale disruption equipment, e.g., high-pressure homogenizers. Rather, the whole cells may be harvested and concentrated by centrifugation (e.g., at about 2° C. to about 4° C.), followed by a water wash to remove residual fermentation broth and an optional second centrifugation. The concentrated whole cells may then be resuspended in cold water to form a concentrated suspension and stored at, for example, about 2° C. to about 4° C.

The concentrated cell suspension may be combined with a buffer including sodium phosphate and MgSO₄ and mixed thoroughly. The resulting solution of yeast cells may have a pH of about 6.5 to about 7.5. The buffer may also contain one or more protease inhibitors, dithiothreitol, and/or glucose. The buffer and any optionally added water may be cooled to about 2° C. to about 4° C. prior to being combined with the concentrated cell suspension. If mechanical cell disruption equipment is used, the equipment may be cooled with ice and recirculating chillers to maintain the equipment at a temperature of about 4° C. during operation. The lysed cells or cell lysate may be collected and, optionally, immediately prepared for fermentation.

### Experimental Test Methods

Seed Fermenter Growth: A Saccharomyces cerevisiae strain, which has deletions in pyruvate decarboxylase genes to restrict the conversion of pyruvate to EtOH and is engineered to produce isobutanol from a carbohydrate source, is grown to 6-7 g/L dcw (2.1×10⁸ cells/mL measured by microscopic cell count) in seed fermenters from a frozen culture, which is stored at −80° C. The culture is grown at 28° C. and the fermenter is agitated at 830 rpm. The seed fermenter medium contains 10 g/L yeast extract, 20 g/L peptone, 25 g/L EtOH, 30 g/L glycerol.

50 mL of the seed fermenter culture is transferred to a second fermenter (1 L). The second fermenter contains 1000 mL of the following medium: 110.0 g/L dextrose, 10 g/L yeast extract, 20 g/L peptone. In the second fermenter, the yeast culture is grown for about 24 hours to about 32 hours in microaerobic conditions followed by growth in anaerobic conditions for about 6 hours to about 14 hours, at pH 5 (maintained using sodium hydroxide), at a temperature of 28° C., and while being agitated at 300 rpm, producing a whole cell batch that includes whole cells and up to about 30 g/L isobutanol. The isobutanol is extracted and the yeast culture is then harvested and concentrated by centrifugation. The cells are washed with cold water, centrifuged again, and then resuspended with 8-10 mL of cold sterile water. The cell suspension is added dropwise to liquid nitrogen to instantly freeze the cells in pellets. The pellets of concentrated frozen yeast cell culture are stored at −80° C. prior to use.

### Cell Lysis

About 4 mL of the concentrated frozen yeast culture is combined with 0.7 mL of a concentrated buffer containing sodium phosphate and MgSO₄ to reach a final solution concentration of 144 mM sodium phosphate and 10 mM MgSO₄ at pH 6.5-7.5. Dithiothreitol and a protease inhibitor is added to the solution of yeast cells and then the solution of yeast cells is manually lysed using a mortar and pestle, which are pre-cooled with liquid nitrogen, for about 3-5 minutes.

### Cell Lysate Fermentation

The fermentation enzymes in the cell lysate are not isolated or separated from the cell debris and 3.7 mL of the cell lysate is directly transferred to a 50 mL flask containing 2.3 mL of the following medium: 200 g/L dextrose, optionally, 9 mL of organic solvent. The cell lysate is allowed to ferment in anaerobic conditions, for about 20 hours to about 25 hours, at about 35° C. to about 40° C. and at a pH ranging from about pH 6.5 to about pH 7.5, in a 225-rpm shaker.

### Example 1. Isobutanol Yield of Cell Lysate Fermentation, with and without an Organic Solvent (Extractant), Versus Isobutanol Yield of Whole Cell Fermentation

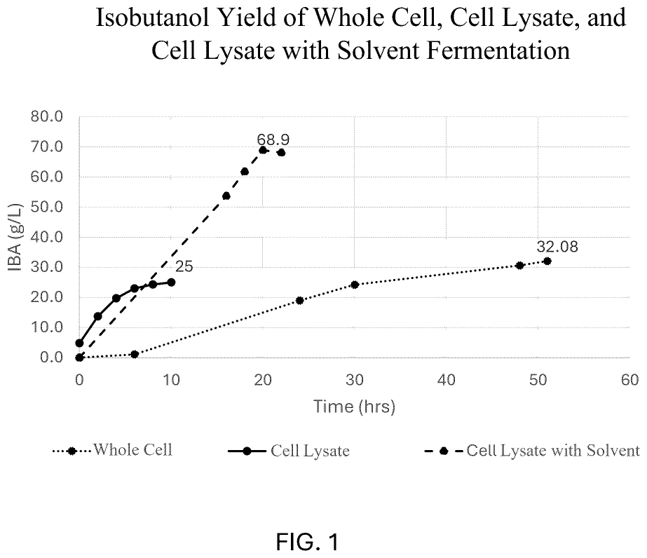

Following fermentation, the isobutanol titer (g/L) is measured for a sample containing whole cells, a sample containing cell lysate, and a sample containing cell lysate and an organic solvent (extractant), all made according to the methods described above. FIG. 1 is a graph showing the isobutanol titers produced by the sample containing whole cells, the sample containing cell lysate, and the sample containing cell lysate and an organic solvent. As shown in the graph of FIG. 1, whole cell batch fermentation, using glucose as the carbon source, produces about 5 g/L of isobutanol in 10 hours and about 30-35 g/L of isobutanol in 50 hours. A cell lysate, which is obtained by harvesting, concentrating, and then lysing the same whole cells, ferments glucose to produce about 25 g/L of isobutanol in 10 hours, under the same batch fermentation conditions. Thus, the cell lysate fermentation produces a greater isobutanol titer in 10 hours than whole cell fermentation, under the same batch fermentation conditions. Without being bound by theory, it is believed that fermentation enzymes in a cell lysate may be more active when the cell lysate is derived from whole cells that are grown in the presence of glucose. It is also believed that because the cell lysate contains all the fermentation enzymes and cofactors that participate in the fermentation, without the constraints of a cell wall and a cell membrane, an increased isobutanol titer may be achieved using cell lysate.

The graph of FIG. 1 also shows that when an organic solvent (extractant) is added to the cell lysate fermentation, even more isobutanol is produced—about 69 g/L of isobutanol is produced in 20 hours. When the organic solvent is added to the cell lysate fermentation broth, a biphasic mixture is formed. In the biphasic mixture, isobutanol partitions into an organic solvent/extractant phase and the fermentation enzymes and glucose partition into an aqueous phase, where the fermentation enzymes remain active. Without being bound by theory, it is believed that alcohol, such as isobutanol, may have a similar inhibitory effect or toxic effect on fermentation enzymes in a cell lysate as it has on cells in whole cell fermentation. For example, the presence of isobutanol may inhibit the conversion of isobutyraldehyde to isobutanol by alcohol dehydrogenase or the presence of isobutanol may compromise enzyme integrity. In the biphasic mixture, fermentation enzymes remain active in the aqueous phase, because the aqueous phase contains a reduced concentration of isobutanol. As such, fermentation reactions continue in the aqueous phase and a greater isobutanol titer is achieved.

### Example 2. Effect of Different Organic Solvents on the Isobutanol Yield of Cell Lysate Fermentation

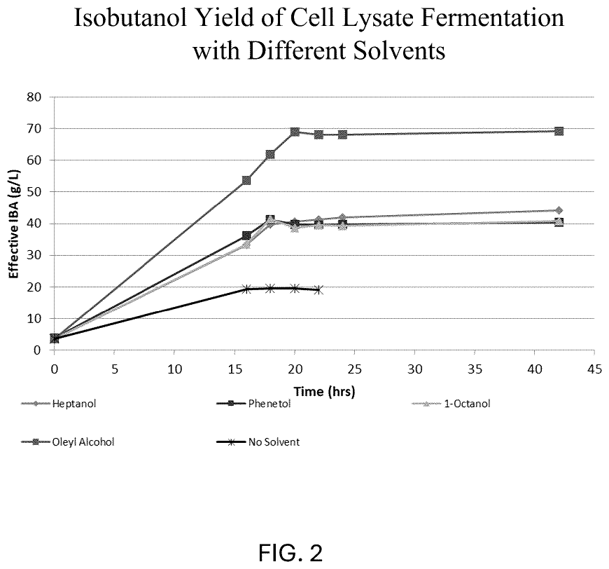

Different organic solvents, namely phenetole, 1-octanol, oleyl alcohol, and heptanol, are added to the cell lysate fermentation broth described in Example 1. The graph of FIG. 2 shows that increased isobutanol titers are achieved with the addition of oleyl alcohol, as compared to the addition of phenetole, 1-octanol, or heptanol. Though, as compared to a control sample that contains no organic solvent, increased isobutanol titers are achieved with the addition of phenetole, 1-octanol, and heptanol.

The foregoing detailed description has been given for clearness of understanding only and no unnecessary limitations should be understood therefrom as modifications will be obvious to those skilled in the art.

While described in connection with specific embodiments thereof, it will be understood that the principles described herein is capable of further modifications and this application is intended to cover any variations, uses, or adaptations following, in general, the principles disclosed herein and including such departures from the present disclosure as come within known or customary practice within the art to which the technology pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

### Exemplary Aspects/Embodiments

Certain aspects, including embodiments/aspects of the present subject matter described above, may be beneficial alone or in combination, with one or more other aspects recited hereinbelow. In addition, while the present subject matter has been disclosed with reference to certain aspects recited below and, in the claims, numerous modifications, alterations, and changes to the described aspects/embodiments are possible without departing from the sphere and scope of the present disclosure. Accordingly, it is intended that the present disclosure is not limited to the described embodiments, aspects, and claims, but that it has the full scope defined by the language of this disclosure and equivalents thereof. While the present technology has been described with reference to the specific aspects/embodiments thereof, it should be understood by those skilled in the art that various changes may be made, and equivalents may be substituted without departing from the true spirit and scope of the disclosure. In addition, many modifications may be made to adapt a particular situation, material, composition of matter, process, and/or process step or steps, to the objective, spirit and scope of the present disclosure. All such modifications are intended to be within the scope of the claims appended hereto.

Herein below are examples of aspects of the present technology.
A. A method of producing renewable alcohol, the method comprising:
   i. providing a recombinant yeast cell comprising an alcohol producing metabolic pathway;
   ii. mixing the recombinant yeast cell with a source of glucose to produce a first reaction mixture and fermenting the first reaction mixture to produce alcohol;
   iii. recovering the alcohol;
   iv. harvesting the recombinant yeast cell;
   v. disrupting the harvested recombinant yeast cell to obtain a substantially cell-free cell lysate;
   vi. mixing the substantially cell-free cell lysate with a source of glucose to produce a second reaction mixture and fermenting the second reaction mixture to produce alcohol; and
   vii. recovering the alcohol.
B. The method according to paragraph A, wherein recovering the alcohol comprises:
   i. combining the first or second reaction mixture and the alcohol with an organic solvent, wherein the alcohol partitions into an organic phase and the first or second reaction mixture partitions into an aqueous phase; and
   ii. distilling the organic phase to separate the alcohol.
C. The method according to any one of paragraphs A-B, wherein the alcohol is recovered before the concentration of the alcohol reaches a level toxic to the recombinant yeast cell.
D. The method according to any one of paragraphs A-C, wherein the alcohol producing metabolic pathway of step a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.
E. The method according to any one of paragraphs A-D, wherein the recombinant yeast cell is disrupted in step b) via homogenization, via cavitation, via one or more cycles of freeze-thawing, or a combination thereof.
F. The method according to any one of paragraphs A-E, wherein the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.
G. The method according to any one of paragraphs A-F, wherein the renewable alcohol is selected from the group consisting of ethanol, 1-butanol, 2-butanol, isobutanol, tert-butanol, and combinations thereof.
H. A method of producing a renewable fuel comprising converting the alcohol produced according to any one of paragraphs A-G to fuel.
I. A method of producing renewable alcohol, the method comprising:
   i. providing a recombinant yeast cell comprising an alcohol producing metabolic pathway;
   ii. disrupting the recombinant yeast cell to obtain a substantially cell-free cell lysate;
   iii. mixing the substantially cell-free cell lysate with a source of glucose to produce a reaction mixture and fermenting the reaction mixture to produce alcohol; and
   iv. recovering the alcohol.
J. The method according to paragraph I, wherein recovering the alcohol comprises:
   i. combining the reaction mixture and the alcohol with an organic extractant, wherein the alcohol partitions into an organic phase and the reaction mixture partitions into an aqueous phase; and
   ii. distilling the organic phase to separate the alcohol.
K. The method according to any one of paragraphs I-J, wherein the alcohol is recovered before the concentration of the alcohol reaches a level toxic to the recombinant yeast cell.
L. The method according to any one of paragraphs I-K, wherein the alcohol producing metabolic pathway of step (a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.
M. The method according to any one of paragraphs I-L, wherein the recombinant yeast cell is disrupted in step (b) via homogenization, via cavitation, via one or more cycles of freeze-thawing, or a combination thereof.
N. The method according to any one of paragraphs I-M, wherein the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.
O. The method according to any one of paragraphs I-N, wherein the renewable alcohol is selected from the group consisting of ethanol, 1-butanol, 2-butanol, isobutanol, tert-butanol, and mixtures thereof.
P. A composition for producing isobutanol comprising a recombinant yeast cell lysate and, optionally, a buffer, wherein the recombinant yeast cell lysate comprises:
   i. an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof; and
   ii. a fragment of recombinant yeast cell wall, a fragment of recombinant yeast cell membrane, or combinations thereof.
Q. The composition according to paragraph P, wherein the recombinant yeast cell lysate is selected from the group consisting of recombinant Saccharomyces cell lysate, recombinant Kluyveromyces cell lysate, recombinant Candida cell lysate, recombinant Pichia cell lysate, recombinant Issatchenkia cell lysate, recombinant Debaryomyces cell lysate, recombinant Hansenula cell lysate, recombinant Yarrowia cell lysate, recombinant Schizosaccharomyces cell lysate, and combinations thereof.
R. A method of producing isobutanol, the method comprising a) mixing the composition according to any one of paragraphs P-Q with a source of glucose to produce a reaction mixture; b) fermenting the reaction mixture to produce isobutanol; and c) recovering the isobutanol.
S. A multiphase fermentation composition comprising:
   i. an aqueous phase comprising the composition according to any one of paragraphs P-Q and a source of glucose; and
   ii. a organic phase comprising isobutanol and an organic solvent.
T. The multiphase fermentation composition of paragraph S, wherein the organic solvent is selected from the group consisting of phenetole, octanol, heptanol, oleyl alcohol, and mixtures thereof.
U. A recombinant yeast cell lysate for producing isobutanol, the recombinant yeast cell lysate made by a method comprising:
   i. providing a recombinant yeast cell comprising an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof;
   ii. mixing the recombinant yeast cell with a source of glucose to produce a first reaction mixture and fermenting the first reaction mixture to produce isobutanol;
   iii. optionally, recovering the isobutanol;
   iv. harvesting the recombinant yeast cell; and
   v. disrupting the harvested recombinant yeast cell to obtain the recombinant yeast cell lysate.
V. A method of producing a recombinant yeast cell lysate, the method comprising:
   i. providing a recombinant yeast cell comprising an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof;
   ii. mixing the recombinant yeast cell with a source of glucose to produce a first reaction mixture and fermenting the first reaction mixture to produce isobutanol;
   iii. optionally, recovering the isobutanol;
   iv. harvesting the recombinant yeast cell; and
   v. disrupting the harvested recombinant yeast cell to obtain the recombinant yeast cell lysate.

What is claimed is:

1. A method of producing renewable alcohol, the method comprising:
a) providing a recombinant yeast cell comprising an alcohol producing metabolic pathway;
b) mixing the recombinant yeast cell with a source of glucose to produce a first reaction mixture and fermenting the first reaction mixture to produce alcohol;
c) recovering the alcohol;
d) harvesting the recombinant yeast cell;
e) disrupting the harvested recombinant yeast cell to obtain a substantially cell-free cell lysate;
f) mixing the substantially cell-free cell lysate with a source of glucose to produce a second reaction mixture and fermenting the second reaction mixture to produce alcohol; and
g) recovering the alcohol.

2. The method according to claim 1, wherein recovering the alcohol comprises:
a) combining the first or second reaction mixture and the alcohol with an organic solvent, wherein the alcohol partitions into an organic phase and the first or second reaction mixture partitions into an aqueous phase; and
b) distilling the organic phase to separate the alcohol.

3. The method according to claim 1, wherein the alcohol is recovered before the concentration of the alcohol reaches a level toxic to the recombinant yeast cell.

4. The method according to claim 1, wherein the alcohol producing metabolic pathway of step a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

5. The method according to claim 1, wherein the harvested recombinant yeast cell is disrupted via homogenization, via cavitation, via one or more cycles of freeze-thawing, or a combination thereof.

6. The method according to claim 1, wherein the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.

7. The method according to claim 1, wherein the renewable alcohol is selected from the group consisting of ethanol, 1-butanol, 2-butanol, isobutanol, tert-butanol, and combinations thereof.

8. A method of producing a renewable fuel comprising converting the alcohol produced according to claim 1 to fuel.

9. A method of producing renewable alcohol, the method comprising:
a) providing a recombinant yeast cell comprising an alcohol producing metabolic pathway;
b) disrupting the recombinant yeast cell to obtain a substantially cell-free cell lysate;
c) mixing the substantially cell-free cell lysate with a source of glucose to produce a reaction mixture and fermenting the reaction mixture to produce alcohol; and
d) recovering the alcohol.

10. The method according to claim 9, wherein recovering the alcohol comprises:
a) combining the reaction mixture and the alcohol with an organic extractant, wherein the alcohol partitions into an organic phase and the reaction mixture partitions into an aqueous phase; and
b) distilling the organic phase to separate the alcohol.

11. The method according to claim 9, wherein the alcohol is recovered before the concentration of the alcohol reaches a level toxic to the recombinant yeast cell.

12. The method according to claim 9, wherein the alcohol producing metabolic pathway of step (a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

13. The method according to claim 9, wherein the recombinant yeast cell is disrupted in step (b) via homogenization, via cavitation, via one or more cycles of freeze-thawing, or a combination thereof.

14. The method according to claim 9, wherein the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.

15. The method according to claim 9, wherein the renewable alcohol is selected from the group consisting of ethanol, 1-butanol, 2-butanol, isobutanol, tert-butanol, and mixtures thereof.

16. A composition for producing isobutanol comprising a recombinant yeast cell lysate and, optionally, a buffer, wherein the recombinant yeast cell lysate comprises:
a) an isobutanol producing metabolic pathway comprising an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof; and
b) a fragment of recombinant yeast cell wall, a fragment of recombinant yeast cell membrane, or combinations thereof.

17. The composition according to claim 16, wherein the recombinant yeast cell lysate is selected from the group consisting of recombinant Saccharomyces cell lysate, recombinant Kluyveromyces cell lysate, recombinant Candida cell lysate, recombinant Pichia cell lysate, recombinant Issatchenkia cell lysate, recombinant Debaryomyces cell lysate, recombinant Hansenula cell lysate, recombinant Yarrowia cell lysate, recombinant Schizosaccharomyces cell lysate, and combinations thereof.

18. A method of producing isobutanol, the method comprising a) mixing the composition according to claim 16 with a source of glucose to produce a reaction mixture; b) fermenting the reaction mixture to produce isobutanol; and c) recovering the isobutanol.

19. A multiphase fermentation composition comprising:
a) an aqueous phase comprising the composition according to claim 16 and a source of glucose; and
b) an organic phase comprising isobutanol and an organic solvent.

20. The multiphase fermentation composition according to claim 19, wherein the organic solvent is selected from the group consisting of oleyl alcohol, phenyl alcohol, cetyl alcohol, lauryl alcohol, myristyl alcohol, stearyl alcohol, oleic acid, lauric acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, isododecanol, lauric aldehyde, 2-methylundecanal, oleamide, linoleamide, palmitamide, stearylamide, 2-ethyl-1-hexanol, 2-hexyl-1-decanol, 2-octyl-1-dodecanol, octanol (e.g., 1-octanol), heptanol, phenetole, and mixtures thereof.
